(19) United States
(12) Patent Application Publication
(10) Pub. No.: US 2018/0292375 A1
(43) Pub. Date: Oct. 11, 2018

(54) METHOD FOR DETECTING BOAR TAINT

(71) Applicant: PHYTRONIX TECHNOLOGIES INC., Quebec, Quebec (CA)

(72) Inventors: Pierre PICARD, Quebec, Quebec (CA); Serge AUGER, Quebec, Quebec (CA)

(73) Assignee: PHYTRONIX TECHNOLOGIES INC., Quebec, Quebec (CA)

(21) Appl. No.: 15/574,316

(22) PCT Filed: Mar. 2, 2017
(86) PCT No.: PCT/CA2017/050281

§ 371 Date: Nov. 15, 2017

Related U.S. Application Data
(60) Provisional application No. 62/302,965, filed on Mar. 3, 2016.

(51) Int. Cl. G01N 33/12; H01J 49/04

(52) U.S. Cl. CPC G01N 33/12; H01J 49/0459; H01J 49/0463

(57) ABSTRACT

A method for detecting boar taint in a fat sample is provided. The method includes extracting boar taint compounds from the fat sample to obtain a boar taint extract which includes indole components and androstenone. The method also includes derivatizing the indole components such that the derivatized indole components have a lower volatility than the indole components. The method also includes desorbing the derivatized indole components and the androstenone by Laser Diode Thermal Desorption (LDTD), and ionizing the desorbed analytes. The content of boar taint compounds in the fat sample can then be determined by subjecting the ionized analytes to mass spectrometry.

### FIELD

The technical field relates to methods for detecting an unpleasant odour or taste in meat, and more particularly relates to methods for detecting boar taint in a fat sample.

### BACKGROUND

Meat from uncastrated boars (including pigs, porks and hogs of the species sus scrofa) develops a fecal and/or urine-like smell. This unpleasant smell is often associated with a bitter taste and poor meat tenderness. As boar taint rarely occurs in castrated boars, male boars were historically castrated at a young age, in order to avoid boar taint. However, the castration of male boars has several disadvantages, such as higher production costs, as well as suffering of the animals. Castrated boars also typically need more time to reach maturity and need to be fed for about two more weeks before being slaughtered. Meat quality originating from castrated boars is lower, as it contains a higher fat-meat ratio. Finally, the European Union has recently decided to ban boar castration by 2018. As hundreds of millions of boars are slaughtered every year for meat consumption, there is a need for methods of detection of boar taint.

Compounds responsible for boar taint include androstenone, indole and skatole. (3-methyl indole). Several methods have been developed to detect these compounds in a fat sample, and are usually based on liquid chromatography or gas chromatography, coupled with mass spectrometry (LC-MS or GC-MS). However, these techniques typically require a long analysis time for each sample which can range from 10 to 30 minutes in the LC or GC columns. Furthermore, LC-MS techniques require the use of solvents which can be costly and/or environmentally unfriendly.

In view of the above, many challenges still exist in the detection of boar taint in fat samples.

### SUMMARY

In some embodiments, a method for detecting boar taint in a fat sample is provided. The method includes extracting boar taint compounds from the fat sample to obtain a boar taint extract which includes indole components and androstenone. The method also includes derivatizing the indole components such that the derivatized indole components have a lower volatility than the indole components. The method also includes drying and desorbing the derivatized indole components and the androstenone by Laser Diode Thermal Desorption (LDTD), and ionizing the desorbed analytes. The content of boar taint compounds in the fat sample can then be determined by subjecting the ionized analytes to mass spectrometry.

In some embodiments, a method for detecting boar taint in a fat sample is provided. The method includes:
extracting boar taint compounds from the animal fat sample, thereby obtaining a boar taint extract comprising indole components and androstenone;
derivatizing the indole components, comprising:
   deprotonating the indole components using a base; and
   alkylating the indole components by reaction with a substrate in a reaction solvent, thereby obtaining solubilized analytes comprising: N-alkylated indole components having a lower volatility than the indole components, and androstenone;
drying the solubilized analytes to obtain dried analytes;
desorbing the dried analytes by Laser Diode Thermal Desorption (LDTD), thereby obtaining desorbed analytes;
ionizing the desorbed analytes, thereby obtaining ionized analytes; and
determining the content of boar taint compounds in the fat sample by subjecting the ionized analytes to mass spectrometry.

In some embodiments, a method for detecting boar taint in a fat sample is provided. The method comprises:
extracting boar taint compounds from the animal fat sample, thereby obtaining a boar taint extract comprising indole components and androstenone;
derivatizing the indole components, comprising:
   deprotonating the indole components using a strong base solubilized in an organic solvent; and
   alkylating the indole components by reaction with a substrate in a reaction solvent, thereby obtaining solubilized analytes comprising: N-alkylated indole components having a lower volatility than the indole components, and androstenone;
drying the solubilized analytes to obtain dried analytes;
desorbing the dried analytes by Laser Diode Thermal Desorption (LDTD), wherein the desorption is induced indirectly by a laser beam without a support matrix and without a liquid mobile phase, thereby obtaining desorbed analytes;
ionizing the desorbed analytes, thereby obtaining ionized analytes; and
determining the content of boar taint compounds in the fat sample by subjecting the ionized analytes to mass spectrometry.

In some embodiments, the fat sample comes from an animal of the species sus scrofa.

In some embodiments, the fat sample is a backfat sample.

In some embodiments, the indole components comprise indole and/or skatole.

In some embodiments, extracting the boar taint compounds from the fat sample comprises liquid-liquid extraction using an extraction solvent.

In some embodiments, the liquid-liquid extraction comprises Salt Assisted Liquid-Liquid Extraction (SALLE).

The method of claim 6, wherein the SALLE comprises:
homogenizing the fat sample in a brine solution;
adding the extraction solvent which is immiscible with the brine solution; and
transferring the boar taint compounds to the extraction solvent.

In some embodiments, the SALLE comprises:
homogenizing the fat sample in a 2-phase system comprising a brine solution and the extraction solvent which is immiscible with the brine solution; and
transferring the boar taint compounds to the extraction solvent.

In some embodiments, the homogenizing comprises at least one of stomaching, sonicating, milling and mixing.

In some embodiments, the mixing comprises vortex mixing.

In some embodiments, mixing the brine solution and the extraction solvent together is followed by centrifuging.

In some embodiments, the extraction solvent comprises at least one of 1-chlorobutane, methyl-ter-butyl ether, diethyl ether, dichloromethane (DCM), chloroform, tetrahydrofuran (THF), ethyl acetate, hexane, acetonitrile, and acetone.

In some embodiments, the extraction solvent comprises acetonitrile.

In some embodiments, the brine solution comprises NaCl.

In some embodiments, the brine solution is a saturated aqueous solution of NaCl.

In some embodiments, the transferring of the boar taint compounds to the extraction solvent comprises mixing the brine solution and the extraction solvent together.

In some embodiments, the method further comprises adding an androstenone internal standard and an indole internal standard to the boar taint extract.

In some embodiments, the androstenone internal standard comprises androstenone-d4.

In some embodiments, the indole internal standard comprises skatole-d3 and/or indole-d7.

In some embodiments, the reaction solvent comprises a polar aprotic solvent.

In some embodiments, the base is a strong base.

In some embodiments, the strong base comprises NaOH or KOH.

In some embodiments, the strong base comprises at least one of sodium bis(trimethylsilyl)amide (NaHMDS), potassium bis(trimethylsilyl)amide (KHMDS) and lithium bis(trimethylsilyl)amide (LiHMDS).

In some embodiments, the strong base is solubilized in a solvent.

In some embodiments, the solvent comprises at least one of THF, hexane, diethyl ether and methyl-ter-butyl ether.

In some embodiments, the solvent is THF.

In some embodiments, the substrate is of general formula R—X, wherein:
R is alkyl, aralkyl, substituted alkyl or substituted aralkyl; and
X is F, Cl, Br, I, OTs, OMs or OTf.

In some embodiments, the substrate is of general formula R—X, wherein:
R is aralkyl; or substituted aralkyl and
X is Cl, Br or I.

In some embodiments, the base is KOH powder and the substrate is benzyl bromide.

In some embodiments, the base is an NaHMDS solution in THF and the substrate is 2,3,4,5,6-pentafluorobenzyl bromide.

In some embodiments, the polar aprotic solvent comprises at least one of acetone, DMF, DMSO and acetonitrile.

In some embodiments, the polar aprotic solvent comprises acetonitrile.

In some embodiments, the reaction solvent and the extraction solvent are the same.

In some embodiments, the extraction solvent is removed prior to adding the reaction solvent.

In some embodiments, drying the solubilized analytes comprises removing the reaction solvent by evaporation at room temperature.

In some embodiments, drying the solubilized analytes comprises removing the reaction solvent by evaporation at atmospheric pressure.

In some embodiments, drying the solubilized analytes comprises removing the reaction solvent by evaporation under vacuum.

In some embodiments, desorbing the dried analytes comprises indirectly heating the dried analytes with infra-red light having a wavelength between 800 and 1040 nm.

In some embodiments, the infra-red light has a power of about 1 to 50 W.

In some embodiments, ionizing the desorbed analytes comprises ionizing using a corona discharge.

In some embodiments, the mass spectrometry comprises tandem mass spectrometry.

### BRIEF DESCRIPTION OF THE DRAWINGS

FIG. 1 is a graph showing measurements of the concentration of androstenone in standard solutions, obtained by a method according to an embodiment in which KOH powder is used as a base;

FIG. 2 is a graph showing measurements of the concentration of skatole in standard solutions, obtained by a method according to an embodiment in which KOH powder is used as a base;

FIG. 3 is a graph showing measurements of the concentration of indole in standard solutions, obtained by a method according to an embodiment in which KOH powder is used as a base;

FIG. 4 is a graph showing measurements of the concentration of androstenone in standard solutions, obtained by a method according to another embodiment in which NaHMDS solubilized in a THF solution is used as a base;

FIG. 5 is a graph showing measurements of the concentration of skatole in standard solutions, obtained by a method according to another embodiment in which NaHMDS solubilized in a THF solution is used as a base; and

FIG. 6 is a graph showing measurements of the concentration of indole in standard solutions, obtained by a method according to another embodiment in which NaHMDS solubilized in a THF solution is used as a base.

### DETAILED DESCRIPTION

The methods described herein pertain to the detection and of boar taint in fat samples. More particularly, the methods described herein can be used for the detection of at least one of the indole compounds responsible for boar taint (i.e., indole and/or skatole) by derivatizing the indole compounds and subjecting the derivatized indole compounds to Laser Diode Thermal Desorption (LDTD) and ionization, and mass spectrometry (also referred to herein as LDTD-MS).

Generally, the method for detecting boar taint, according to embodiments of the present description, first includes an extraction of boar taint compounds from the fat sample to obtain a boar taint extract which includes indole components (such as indole and/or skatole) and androstenone. The method also includes derivatizing indole components to obtain compounds having a lower volatility than the indole components. The method also includes drying and desorbing the derivatized indole components and the androstenone by Laser Diode Thermal Desorption (LDTD), and ionizing the desorbed analytes. The method further includes determining the content of boar taint compounds by subjecting the ionized analytes to mass spectrometry.

The methods described herein may generally be useful in any application where it is desired to detect volatile compounds using LDTD-MS, which can be derivatized prior to being desorbed to lower their volatility. The derivatized compounds can then be ionized and subjected to mass spectrometry. It is understood that while the present description aims at describing methods for the detection of boar taint compounds in fat samples, the methods described herein are also applicable to other compounds which can be derivatized, desorbed and ionized in a manner which is similar to that of the indole compounds of the boar taint compounds. It is also understood that the desorption using the LDTD technique is induced indirectly by a laser beam without a support matrix (unlike the MALDI technique) and without a liquid mobile phase, and that ionization may be achieved by a corona discharge. It is understood that carrying out the ionization without a liquid mobile phase differentiates this technique from the standard APCI technique which is typically carried with at least traces of solvent present. Typically, the ionization used in conjunction with the LDTD technique is performed in an environment which is mostly free of mobile phase or solvent, but it is understood that traces of moisture (such as moisture present in the ambient air) can be present during ionization. In some embodiments, UV radiations may be used to complement the corona discharge as an ionizing means. It is therefore understood that LDTD is matrix and mobile phase free, and may thereby eliminate cross contamination of samples. In some embodiments, the methods described herein may have the advantage of allowing a fat sample to be derivatized in a few minutes and then analyzed in a few seconds (in some instances, from 5 to 60 seconds), as opposed to 10 to 30 minutes for known techniques such as LC-MS and GC-MS.

It is understood that the LDTD techniques of the present disclosure refer to the techniques described in U.S. Pat. Nos. 7,321,116 and 7,582,863, the contents of which are hereby incorporated by reference in their entirety.

It is understood that the term “boar taint” refers to the offensive odor or taste which can arise during the cooking or eating of boars or boar products derived from non-castrated male boars (including pigs, porks and hogs of the species sus scrofa) once they reach puberty. It is generally known that “boar taint” is caused by the accumulation of androstenone and skatole (3-methyl indole)—and of indole to a lesser degree—in the fat of a minor number of male boars. Androstenone is a male pheromone which is produced in the testes as male boars reach puberty, while skatole and indole are produced in both male and female boars. Skatole and indole are a byproduct of intestinal bacteria, or bacterial metabolite of the amino acid tryptophan. It is also generally known that the skatole and indole levels are much higher in uncastrated boars, because testicular steroids inhibit the breakdown of skatole and indole (also referred to as indole components) by the liver, which causes accumulation of these compounds in the fat, as the male boars mature.

It is understood that by “detecting” or “detection” of an analyte, it is meant that a concentration of the analyte producing a signal which is greater than the instrument detection limit is measured (i.e., a concentration greater than three times the standard deviation of the noise level is measured). It is also understood that by “detecting” or “detection” of boar taint in a fat sample, it is meant that at least one of the compounds responsible for boar taint (i.e., androstenone, skatole or indole) has a measured concentration that is greater than a maximum concentration which is set by national or regional thresholds.

It is understood that the term “fat sample” refers to a sample from an animal carcass. For example, in the context of the present disclosure, the fat sample can originate from an animal of the species sus scrofa which includes boars, pigs, porks and hogs. The fat sample can originate from adipose tissue of an animal (i.e. fat of an animal). For example, one of the adipose tissue which is typically used to analyse the level of boar taint compounds is the subcutaneous fat from the dorsal mid-loin site in a boar carcass (also referred to as backfat). It is understood that fat samples (or meat samples which include fat) originating from other parts of the carcass can be used to detect boar taint, such as meat samples or fat samples from the neck and cheek.

In some embodiments, the method includes extracting boar taint compounds from the fat sample, in order to obtain an extract comprising indole components and androstenone (also referred to herein as boar taint extract). It is understood that the terms “extracting” or “extraction” refer to a separation process which aims at separating a substance or several substances from a matrix. In some embodiments, the extraction includes liquid-liquid extraction (or solvent extraction). An example of liquid-liquids extraction which can be used is Salt Assisted Liquid-Liquid Extraction (SALLE). It is understood that SALLE refers to an extraction process in which an inorganic salt is present or added into a mixture of water and a water-miscible organic solvent, and in which the inorganic salt causes the separation of the water-miscible solvent from the mixture, with formation of a two-phase system. SALLE can sometimes be referred to as “salt-induced phase separation”. Extraction solvents which can be used in SALLE include but are not limited to acetone, isopropanol, and/or acetonitrile. It is also understood that different inorganic salts and different inorganic salt concentrations can be used. Brine can be used as a salt-containing aqueous solution for SALLE. It is understood that the term “brine” refers to a solution of salt which has a concentration of salt ranging from about 3.5 wt % to saturation. For example, NaCl may be used as the inorganic salt, and saturated NaCl solutions may be used as the salt-containing aqueous solution for SALLE.

In some embodiments, the liquid-liquid extraction includes homogenizing the fat sample in a solution. It is understood that the term “homogenization” refers to a process in which the fat sample is turned into small particles of fat distributed uniformly throughout the solution. In some embodiments, homogenization is performed using at least one of stomaching, sonicating (such as focus sonicating), milling and mixing (such as vortex mixing). In some embodiments, the solution is an aqueous solution such as a brine solution. In some embodiments, an extraction solvent which is immiscible with the brine solution is added, and the boar taint compounds are transferred to the extraction solvent. In other embodiments, an extraction solvent which is miscible with the aqueous solution (which is not a brine solution) is added, and an inorganic salt is subsequently added to the mixture to separate the mixture in two phases (including an aqueous phase including the inorganic salt, and the extraction solvent). In other embodiments, the solution comprises an aqueous solution and an extraction solvent which may or may not be miscible with the aqueous solution, and the fat sample is homogenized directly in the mixture.

In some embodiments, the fat sample can be homogenized in a 2-phase system comprising an aqueous solution (such as brine) and an organic solvent (such as acetonitrile). After homogenization in the 2-phase system, the homogenized fat sample is typically in the form of fat particles dispersed in the 2-phase system. When the aqueous solution is brine and the organic solvent is a polar organic solvent which is immiscible with the brine (e.g. acetonitrile), the fat particles can be transferred to the organic solvent and dispersed therein as a result of the homogenization.

In some embodiments, the extraction is a liquid-liquid extraction which can include:
homogenizing the fat sample in an aqueous solution;
adding an extraction solvent which is immiscible with the aqueous solution; and
transferring the boar taint compounds to the extraction solvent.

In some embodiments, the extraction is a SALLE which can include:

homogenizing the fat sample in an aqueous solution;
adding an extraction solvent which is miscible with the aqueous solution;
adding an inorganic salt to the mixture, thereby separating the mixture into an organic phase and an aqueous phase; and
transferring the boar taint compounds to the extraction solvent.

In some embodiments, the extraction is a SALLE which can include:
homogenizing the fat sample in a brine solution;
adding an extraction solvent which is immiscible with the brine solution; and
transferring the boar taint compounds to the extraction solvent.

In some embodiments, the extraction is a SALLE which can include:
homogenizing the fat sample in a mixture comprising a brine solution and an extraction solvent (e.g. acetonitrile) which is immiscible with the brine solution; and
transferring the boar taint compounds to the extraction solvent.

It is understood that when the homogenization of the fat sample is performed directly in a mixture comprising a brine solution and an extraction solvent which is immiscible with the brine solution, the transfer of the boar taint compounds to the extraction solvent can happen directly as a result of the homogenization, without the need of further extraction steps.

In some embodiments, the extraction solvent includes at least one of dichloromethane, chloroform, 1-chlotobunate, diethyl ether, methyl-ter-butyl ether, tetrahydrofuran, ethyl acetate, acetonitrile, isopropanol, and acetone. In some scenarios where the extraction is a SALLE, the extraction solvent can include at least one of acetonitrile, isopropanol and acetone. In some embodiments, transferring the boar taint compounds to the extraction solvent comprises mixing the aqueous solution and the extraction solvent together. In some embodiments, mixing the aqueous solution and the extraction solvent together can be followed by centrifuging.

In some embodiments, the extraction can include homogenizing the fat sample in a mixture comprising an aqueous solution (e.g. water) and an organic solvent which may be miscible with water (e.g. methanol, ethanol, acetonitrile, acetone, and/or isopropanol), or immiscible with water (e.g. dichloromethane, chloroform, 1-chlotobunate, diethyl ether, methyl-ter-butyl ether, tetrahydrofuran, ethyl acetate). The extraction can further include centrifuging the mixture, thereby precipitating unwanted material such as proteins. The precipitated material can be discarded, and the liquid phase which includes the boar taint compounds can be recovered.

In some embodiments, the indole components included in the boar taint extract can be derivatized in order to obtain derivatized indole components which have a lower volatility than the indole components. It is understood that the term “derivatization” as used herein refers to a chemical reaction which transforms a chemical compound into a derivate in which a specific functional group of the compound is transformed so as to modify a certain physical and/or chemical property of the compound. For instance, the derivatization reactions which can be used in the methods of the present description can allow for a reduction in the volatility of the indole components. It is understood that several characteristics may be desirable for a derivatization reaction to be used in the methods described herein, such as:
(i) a reaction which proceeds to completion;
(ii) a reaction which may be used substantially as efficiently on a wide range of compounds (e.g. the indole components—skatole and indole—as well as indole internal standards); and
(iii) a reaction which yields derivatized products which are relatively stable and form no degradation products within a reasonable period, so as to facilitate the analysis.

Examples of derivatization reactions which may be used include one of acylation, alkylation, and protonation of the indole component —NH group. For example, the acylation reaction can include acylating the —NH group of the indole components using an anhydride, such as trifluoroacetic anhydride (TFAA), heptafluorobutyric acid (HFBA), Heptafluorobutyryl imidazole (HFBI), N-methyl-bis(heptafluorobutyramide) (MBHFBA), or pentafluoropropionic anhydride (PFPA). For example, the protonation of the indole component —NH group can include reacting the indole component with a strong acid such as hydrochloric acid for salt formation. For example, the alkylation of the indole component —NH group can include subjecting the indole component to a nucleophilic substitution reaction using a base to deprotonate the —NH group, followed by reacting the indolate base thereby obtained with an electrophile including a leaving group. It is understood that several derivatization which are described in the “Handbook of analytical derivatization reactions” (D. R. Knapp), may be used in certain embodiments of the present description, so long as the volatility of the derivatized compounds is lower than the volatility of the non-derivatized analytes.

It is understood that the term “volatility” as used herein refers to the tendency of a substance to vaporize. The volatility is directly related to the substance's vapor pressure, i.e. at a given temperature, a substance with higher vapor pressure vaporizes more readily than a substance with a lower vapor pressure. It is therefore understood that the derivatization reaction performed to “lower the volatility” of the indole components allows to obtain derivatized indole components which have a lower tendency to vaporize than the indole components.

In some embodiments, the derivatization of the indole components may include deprotonating the indole components using a base, and alkylating the deprotonated indole components with a substrate. Alkylating of the deprotonated indole component can take place in a polar aprotic solvent. In some embodiments, the base is a strong base, such as NaOH, KOH, NaH, KH, butyl lithium, sodium bis(trimethylsilyl)amide (NaHMDS), potassium bis(trimethylsilyl)amide (KHMDS) or lithium bis(trimethylsilyl)amide (LiHMDS). The base can be used in powder form, or can be used in solution in a solvent. An example of a base in powder form is powder NaOH or KOH. An example of a base in solution in a solvent is NaHMDS in THF.

It was found that for the purpose of automated analyses, in which a high number or samples are to be analyzed every hour, the use of a strong base solubilized in a solvent is typically preferred to a strong base in solid or powder form, as a strong base in solid or powder form (such as solid NaOH or KOH) may absorb more water than a base solubilized in a solvent. In other words, the use of a base solubilized in a solvent may not require the use of anhydrous conditions for deprotonating the indole components. In some embodiments, the solvent used to solubilize the base is an organic solvent which can include at least one of THF, hexane, diethyl ether and methyl-tert-butyl ether. In some scenarios, the use of a strong base solubilized in an organic solvent (such as NaHMDS, KHMDS or LiHMDS in THF), may be preferred over a base in solid or powder form (such as KOH or NaOH in powder form).

In some embodiments, the substrate is of general formula R—X, wherein:
R is alkyl, aralkyl, substituted alkyl or substituted aralkyl; and
X is F, Cl, Br, I, OTs, OMs or OTf, wherein OTs refers to tosylate, OMs refers to mesylate, and OTf refers to triflate,
   with the limitation that the alkylated indole components have a lower volatility than the indole components. Are therefore excluded substrates which will yield alkylated indole components having a higher volatility than the indole components. An example of an excluded substrate is methyl iodide, since the N-methyl indole and N-methyl skatole are known to have a higher volatility than indole and skatole, respectively.

In some embodiments, the substrate is of general formula R—X, wherein:
R is aralkyl; or substituted aralkyl and
X is Cl, Br or I.

It is understood that the term “alkyl”, as used herein, refers to linear, branched or cyclic saturated monovalent hydrocarbon radicals or a combination of cyclic and linear or branched saturated monovalent hydrocarbon radicals which have 1 or more carbon atoms. Examples of “alkyl” include, but are not limited to, methyl, ethyl, n-propyl, isopropyl, isobutyl, n-butyl, sec-butyl, tert-butyl, isopentyl, n-pentyl, neopentyl, n-hexyl, and 2-ethylhexyl.

It is understood that the term “aralkyl”, as used herein, refers to an alkyl group which is substituted with an aryl group. Aralkyl groups include, but are not limited to benzyl and picolyl groups.

It is understood that the terms “substituted” refers to substitution of one or more hydrogens of the designated moiety or group with a substituent or substituents, multiple degrees of substitutions being allowed unless otherwise stated, and provided that the substitution results in a stable or chemically feasible compound. For example, the substituents may be one or multiple halogens (such as fluoride). For example, a substituted aralkyl group may be 2,3,4,5,6-pentafluorobenzyl.

In some embodiments, the pair base/substrate is selected such that the derivatization reaction proceeds to completion and is completed within a certain time. For example, in some embodiments, the base is KOH powder or NaOH powder, and the substrate is a benzyl bromide or a substituted benzyl bromide. In another example, the base is an NaHMDS, KHMDS or LiHMDS solution in THF, and the substrate is benzyl bromide or a substituted benzyl bromide such as 2,3,4,5,6-pentafluorobenzyl bromide.

In some embodiments, the polar aprotic solvent includes at least one of acetone, DMF, DMSO and acetonitrile. It is understood that the polar aprotic solvent can be the same solvent as the extraction solvent if the extraction solvent which is used in the extraction step has the properties required for the derivatization reaction to be conducted in it. It is also understood that the polar aprotic solvent can be a different solvent as the extraction solvent. In such case, the extraction solvent in which the boar taint compounds are transferred in the extraction step can be removed, and the polar aprotic solvent can subsequently be added to solubilize the solid residue, and the derivatization reaction can be conducted.

In some embodiments, the method further includes extracting the derivatized indole components from the reaction solvent using a second extraction solvent which includes an apolar organic solvent. The apolar organic solvent may include at least one of ethyl acetate, 1-Chlorobutane, methyl-ter-butyl ether, dichloromethane, chloroform, hexane, pentane, heptane, petroleum ether, benzene, toluene, diethyl ether and 1,4-dioxane. For example, the apolar organic solvent can include a mixture of ethyl acetate and hexane in a ratio between 10/90 and 90/10 v/v.

In some embodiments, the method further includes adding an androstenone internal standard and an indole internal standard to the boar taint extract. It is understood that the term “internal standard” refers to a chemical substance which is added in a known amount to samples, the blank and the calibration standards in a chemical analysis. This chemical substance can then be used for calibration by plotting the ratio of the analyte signal to the internal standard as a function of the analyte concentration of the standards. For example, the use of an internal standard can be used to correct for the loss of analyte during sample preparation, such as during the extraction step and/or the derivatization step. It is also understood that the internal standard is a compound which is of similar nature than the compounds to be analyzed in the sample, without being identical to the compounds to be analyzed, so that the effects of sample preparation can be taken into account upon measuring concentrations of the compounds. For example, a suitable androstenone internal standard is androstenone-d4, androstenone-d5 or a C¹³-labeled androstenone, and a suitable internal standard for skatole and indole can be skatole-d3 and/or indole-d7, or a C¹³-labeled skatole or indole. In some scenarios, a single internal standard is used for both skatole and indole. It is also understood that the internal standards mentioned herein are non-limiting, and that other internal standards may be used. In some embodiments, the androstenone internal standard and the indole internal standard are added at the beginning of the extraction step, for example after the fat sample has been homogenized in the aqueous solution. In some embodiments, the internal standard can be used as a “cut off” reference. For example, the concentration of internal standard added can correspond to a concentration limit which, if exceeded, can result in discarding the carcass from which the fat sample originated.

It is understood that prior to desorbing the derivatized indole components and the androstenone, the solvent in which the derivatized indole components and the androstenone are solubilized is removed. In other words, in some embodiments, the method further includes drying the solubilized analytes to obtain dried analytes. In some embodiments, drying the solubilized analytes includes removing the solvent in which the derivatized indole components and the androstenone are solubilized by evaporation of the solvent at room temperature and/or atmospheric pressure. In some embodiments, drying the solubilized analytes includes removing the solvent under vacuum. It is understood that the solvent to be removed can be the reaction solvent or the second extraction solvent in cases where the derivatized indole components and the androstenone are extracted from the reaction solvent prior to being dried.

Desorbing the derivatized indole components and the androstenone by LDTD includes indirectly heating the derivatized components and the androstenone with infra-red light, such as infra-red light having a wavelength between 800 and 1040 nm. In some embodiments, the infra-red light has a power of about 1 to 50 W.

In some embodiments, ionizing the desorbed analytes includes ionizing using a corona discharge.

It is understood that the term “mass spectrometry” as used herein refers to analytical techniques which allow identifying chemicals present in a sample by measuring the mass-to-charge ratio and abundance of gas-phase ions. In some embodiments, the mass spectrometry includes tandem mass spectrometry. It is understood that the term “tandem mass spectrometry” refers to the use of a mass spectrometer which makes use of two or more mass analyzers. The mass spectrometers which may be used in the methods of the present description include, for example a Time-of-fight mass spectrometer, a quadrupole mass analyzer, a quadrupole ion trap, a cylindrical ion trap, a linear quadrupole ion trap and/or an orbitrap.

It is understood that the scope of the claims should not be limited by the preferred embodiments set forth herein, but should be given the broadest interpretation consistent with the description as a whole.

### EXAMPLES

### Example 1

Experiments were conducted to prepare standard solutions of known concentrations of androstenone, skatole or indole. The standard solutions were prepared by SALLE of minced pig backfat samples, as follows:
0.5 g of minced pig backfat which did not contain boar taint compounds was homogenized in a mixture of 1.5 mL of saturated NaCl solution and 1.5 mL of an internal standard solution of skatol-d3 (50 ppb) and androstenone-d4 (500 ppb) in acetonitrile;
the mixture obtained was vortex mixed and centrifuged, and the upper fraction (acetonitrile fraction) was collected;
a known concentration of androstenone, skatole and indole was added in each standard solution.

The concentrations in androstenone, skatole and indole are shown in Tables 1 to 3 for each one of the standard solutions prepared. Skatole-d3 was used as the internal standard for both skatole and indole.

TABLE 1
Standard solutions of known concentrations of androstenone,
skatole and indole

| | | Skatole | Indole |
|---|---|---|---|
| Standard | Androstenone | concentration | concentration |
| solution # | concentration (ppb) | (ppb) | (ppb) |
| STD1 | 200 | 50 | 10 |
| STD2 | 400 | 100 | 20 |
| STD3 | 1000 | 250 | 50 |
| STD4 | 2000 | 500 | 100 |
| STD5 | 4000 | 1000 | 200 |

The standard solutions listed in Table 1 were then analyzed using methods according to embodiments of the present description, and calibration curves were plotted.

### Example 2

Experiments were conducted to measure the concentration of androstenone, skatole and indole in the standard solutions listed in Table 1 of Example 1, using a method according to an embodiment of the present description.

Each standard solution was submitted to the following derivatization procedure:
10 to 20 mg of KOH powder was added to 100 μl of the standard solution in anhydrous conditions (acetonitrile fraction of Example 1 to which androstenone, skatole and indole were added);
10 μl of a benzyl bromide solution (10% v/v in acetonitrile) was added;
the mixture obtained was vortex mixed and the reaction was allowed to go on for 5 minutes at room temperature;
400 μl of an EDTA buffer (0.25M, pH 8) was added;
400 μl of a hexane/ethyl acetate mixture (90/10 v/v) was added;
the mixture was vortex mixed and the two phases were allowed to separate;
5 μl of the upper layer phase was deposited onto a LazWell™ well surface and allowed to dry at room temperature.

Each dried sample was then subjected to LDTD-MS/MS using a LDTD™ S-960 model and an AB Sciex 5500 QTRAP™ tandem mass spectrometer. The carrier gas was air, used at 3 L/min. The ionization mode was set to positive mode. Each measurement was reproduced four times.

The laser pattern used to desorb each dried sample was as follows:
0% laser power from t=0 s to t=1.0 s;
linearly ramping from 0% laser power to 35% laser power from t=1 s to t=7.0 s;
35% laser power from t=7.0 s to t=9.0 s;
0% laser power from t=9.0 s to 10.0 s.

The m/z ratios and collision energy obtained for each compound are shown in Table 2.

TABLE 2
m/z ratios and collision energy

| Compound | Q1 | Q3 | Collision energy |
|---|---|---|---|
| Androstenone | 273 | 215 | 25 |
| Androstenone-d4 | 277 | 215 | 25 |
| Skatole | 222 | 91 | 25 |
| Indole | 208 | 91 | 25 |
| Skatole-d3* | 225 | 91 | 25 |

*Skatole-d3 was used as internal standard for indole.

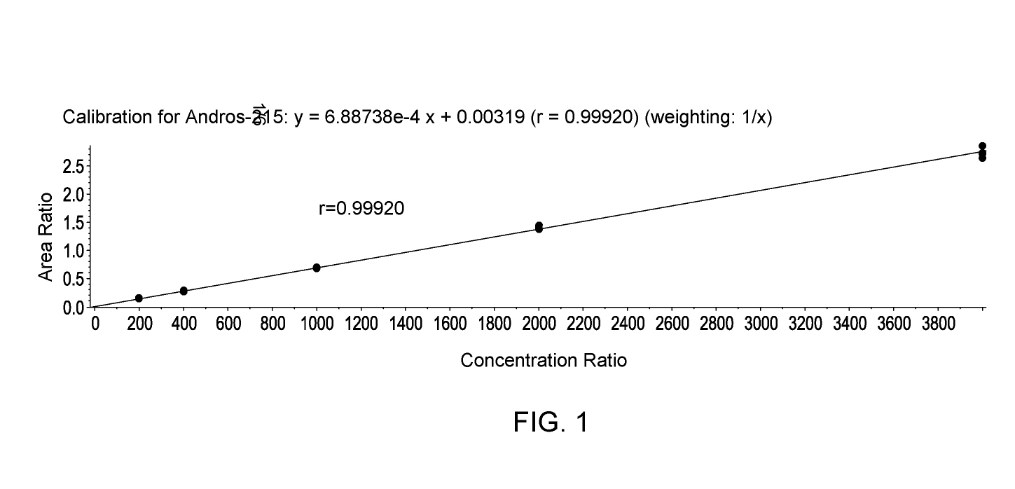

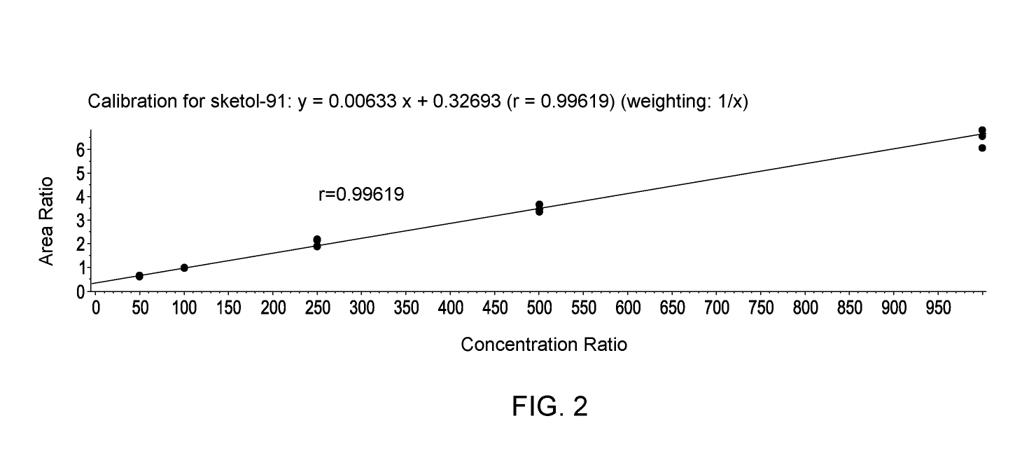

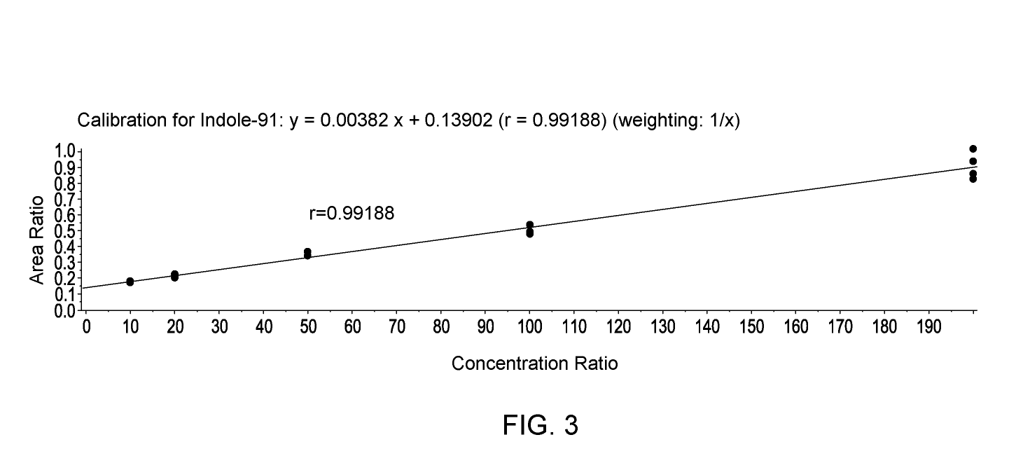

The results for the measured concentrations of androstenone, stakole and indole in the standard solutions STD1-5 are shown in Tables 3-5, and the calibration curve is shown on FIGS. 1-3.

TABLE 3
measurements of androstenone concentrations in standard
solutions STD1-5

| | | | Mean | | | |
|---|---|---|---|---|---|---|
| Standard | Concentration | | measurement | | | |
| solution # | (ppb) | N | (ppb) | SD | % CV | % Nom |
| STD1 | 200 | 4 | 204.9 | 12.8 | 6.2 | 102.5 |
| STD2 | 400 | 4 | 387.5 | 16.0 | 4.1 | 96.9 |
| STD3 | 1000 | 4 | 987.6 | 11.6 | 1.2 | 98.8 |
| STD4 | 2000 | 4 | 2056.3 | 45.8 | 2.2 | 102.8 |
| STD5 | 4000 | 4 | 3963.7 | 133.4 | 3.4 | 99.1 |

TABLE 4
measurements of skatole concentrations in standard
solutions STD1-5

| | | | Mean | | | |
|---|---|---|---|---|---|---|
| Standard | Concentration | | measurement | | | |
| solution # | (ppb) | N | (ppb) | SD | % CV | % Nom |
| STD1 | 50 | 4 | 46.4 | 7.5 | 16.2 | 92.7 |
| STD2 | 100 | 4 | 100.6 | 3.1 | 3.1 | 100.6 |
| STD3 | 250 | 4 | 269.6 | 24.6 | 9.1 | 107.8 |
| STD4 | 500 | 4 | 505.2 | 23.7 | 4.7 | 101.0 |
| STD5 | 1000 | 4 | 971.1 | 61.2 | 6.3 | 97.1 |

TABLE 5
measurements of indole concentrations in standard
solutions STD1-5

| | | | Mean | | | |
|---|---|---|---|---|---|---|
| Standard | Concentration | | measurement | | | |
| solution # | (ppb) | N | (ppb) | SD | % CV | % Nom |
| STD1 | 10 | 4 | 10.0 | 2.0 | 20.3 | 100.2 |
| STD2 | 20 | 4 | 19.4 | 3.3 | 17.0 | 97.2 |
| STD3 | 50 | 4 | 56.8 | 4.7 | 8.3 | 113.5 |
| STD4 | 100 | 4 | 94.7 | 7.1 | 7.5 | 94.7 |
| STD5 | 200 | 4 | 202.5 | 22.4 | 11.0 | 101.2 |

The derivatization of indole and skatole decreased the volatility of the compounds, which allowed measuring their concentration by LDTD-MS/MS. The calibration curves obtained and seen on FIGS. 1-3 are linear and show reproducibility of the method used. The precision (% CV) was found to be between 1.2 and 20.3%, and the accuracy (% Nominal) to be between 92.7 and 113.5%.

It was found that using KOH in solution in water did not allow for the derivatization to take place.

It was also found that using a KOH powder in ambient conditions (i.e., non-anhydrous conditions or conditions where the KOH powder is left exposed to the ambient atmosphere for several minutes) decreased the efficiency of the derivatization.

### Example 3

Experiments were conducted to measure the concentration of androstenone, skatole and indole in the standard solutions listed in Table 1 of Example 1, using a method according to another embodiment of the present description.

Each standard solution was submitted to the following derivatization procedure:
20 μl of a sodium bis(trimethylsylil) amide (NaHMDS) solution in THF (1.0M) was added to 100 μl of the standard solution (acetonitrile fraction of Example 1 to which androstenone, skatole and indole were added) in ambient conditions;
The mixture was vortex mixed;
10 μl of a 2,3,4,5,6-pentafluorobenzyl bromide solution (10% v/v in acetonitrile) was added;
the mixture obtained was vortex mixed and the reaction was allowed to go on for 5 minutes at 37° C.;
400 μl of a hexane/ethyl acetate mixture (90/10 v/v) was added;
the mixture was vortex mixed and the two phases were allowed to separate;
2 μl of the upper layer phase was deposited onto a LazWell™ well surface and allowed to dry at room temperature.

Each dried sample was then subjected to LDTD-MS/MS using a LDTD™ S-960 model and an AB Sciex 5500 QTRAP™ tandem mass spectrometer. The carrier gas was air, used at 3 L/min. The ionization mode was set to positive mode. Each measurement was reproduced three times.

The laser pattern used to desorb each dried sample was as follows:
0% laser power from t=0 s to t=1.0 s;
linearly ramping from 0% laser power to 35% laser power from t=1 s to t=7.0 s;
35% laser power from t=7.0 s to t=9.0 s;
0% laser power from t=9.0 s to 10.0 s.

The m/z ratios and collision energy obtained for each compound are the same as the values obtained in Table 2 of Example 2.

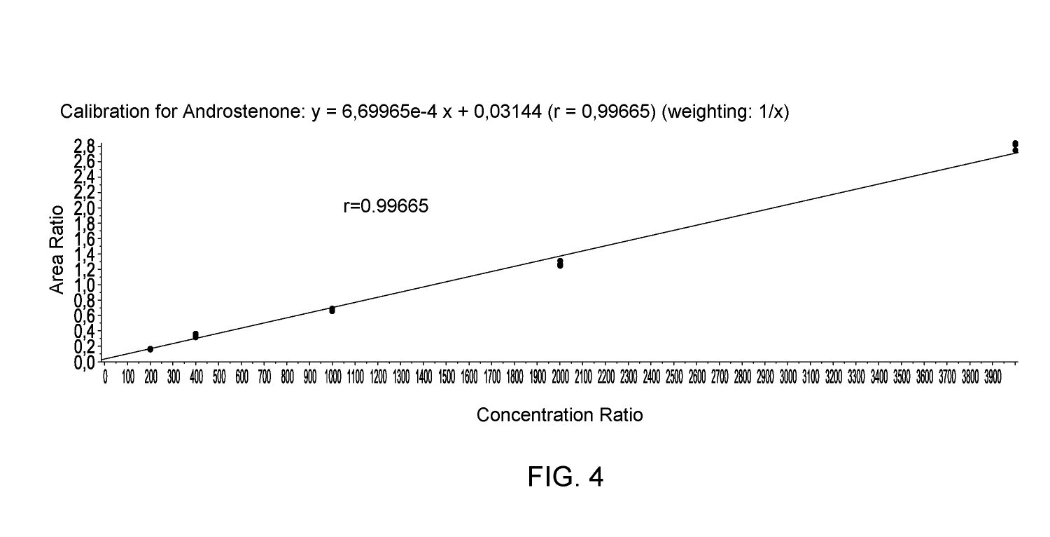

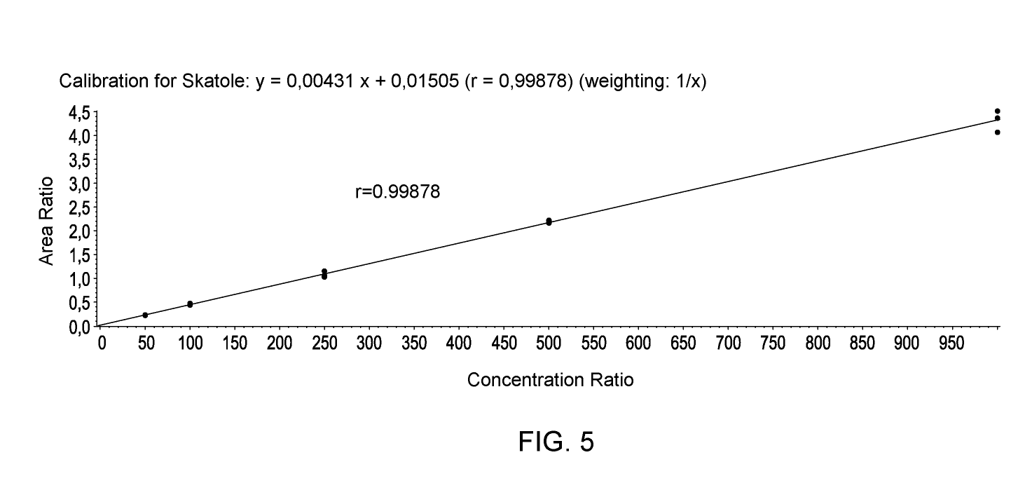

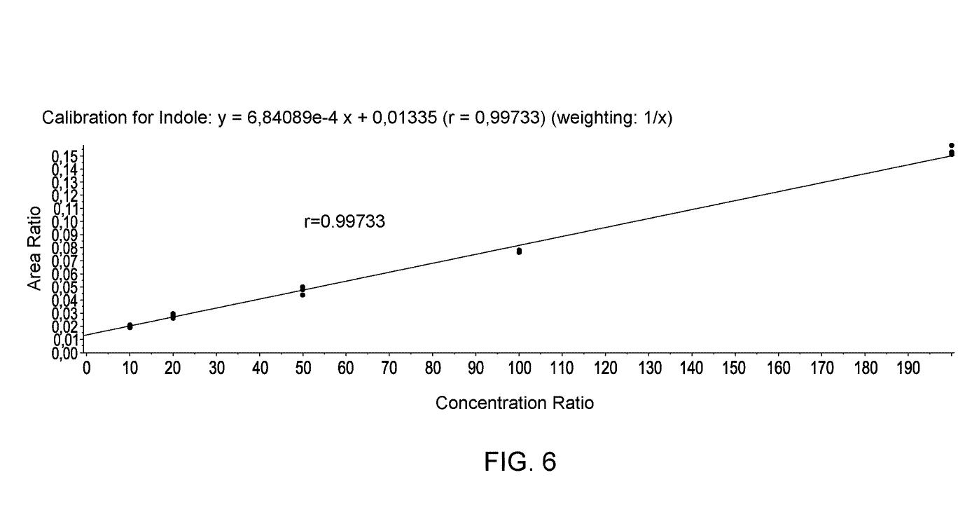

The results for the measured concentrations of androstenone, stakole and indole in the standard solutions STD1-5 are shown in Tables 6-8, and the calibration curve is shown on FIGS. 4-6.

TABLE 6
measurements for androstenone standard solutions

| | | | Mean | | | |
|---|---|---|---|---|---|---|
| Standard | Concentration | | measurement | | | |
| solution # | (ppb) | N | (ppb) | SD | % CV | % Nom |
| STD1 | 200 | 3 | 183.5 | 12.7 | 6.9 | 91.7 |
| STD2 | 400 | 3 | 464.7 | 28.8 | 6.2 | 116.2 |
| STD3 | 1000 | 3 | 958.8 | 31.8 | 3.3 | 95.9 |
| STD4 | 2000 | 3 | 1855.1 | 44.9 | 2.4 | 92.8 |
| STD5 | 4000 | 3 | 4138.0 | 75.5 | 1.8 | 103.5 |

TABLE 7
measurements for skatole standard solutions

| | | | Mean | | | |
|---|---|---|---|---|---|---|
| Standard | Concentration | | measurement | | | |
| solution # | (ppb) | N | (ppb) | SD | % CV | % Nom |
| STD1 | 50 | 3 | 48.7 | 1.2 | 2.4 | 97.5 |
| STD2 | 100 | 3 | 103.5 | 3.9 | 3.8 | 103.5 |
| STD3 | 250 | 3 | 245.9 | 15.1 | 6.1 | 98.4 |
| STD4 | 500 | 3 | 505.0 | 7.5 | 1.5 | 101.0 |
| STD5 | 1000 | 3 | 996.9 | 53.5 | 5.4 | 99.7 |

TABLE 8
measurements for indole standard solutions

| | | | Mean | | | |
|---|---|---|---|---|---|---|
| Standard | Concentration | | measurement | | | |
| solution # | (ppb) | N | (ppb) | SD | % CV | % Nom |
| STD1 | 10 | 3 | 9.9 | 1.5 | 14.8 | 99.1 |
| STD2 | 20 | 3 | 21.0 | 2.4 | 11.5 | 105.1 |
| STD3 | 50 | 3 | 49.6 | 4.5 | 9.0 | 99.3 |
| STD4 | 100 | 3 | 93.8 | 1.2 | 1.3 | 93.8 |
| STD5 | 200 | 3 | 205.7 | 5.2 | 2.5 | 102.9 |

The derivatization of indole and skatole decreased the volatility of the compounds, which allowed measuring their concentration by LDTD-MS/MS. The calibration curves obtained and seen on FIGS. 4-6 are linear and show reproducibility of the method used. The precision (% CV) was found to be between 1.3 and 14.8%, and the accuracy (% Nominal) to be between 91.7 and 116.2%.

Compared to the use of KOH in powder form, it was found that the use of a strong base (in this Example, the strong organic base NaHMDS) in an organic solvent (in this Example, THF) is preferred, in that anhydrous conditions were not required in order to achieve an efficient derivatization.

1.-34. (canceled)

35. A method for detecting boar taint in a fat sample, comprising:
extracting boar taint compounds from the animal fat sample, thereby obtaining a boar taint extract comprising indole components and androstenone;
derivatizing the indole components, comprising:
deprotonating the indole components using a strong base solubilized in an organic solvent; and
alkylating the indole components by reaction with a substrate in a reaction solvent, thereby obtaining solubilized analytes comprising: N-alkylated indole components having a lower volatility than the indole components, and androstenone;
drying the solubilized analytes to obtain dried analytes;
desorbing the dried analytes by Laser Diode Thermal Desorption (LDTD), wherein the desorption is induced indirectly by a laser beam without a support matrix and without a liquid mobile phase, thereby obtaining desorbed analytes;
ionizing the desorbed analytes, thereby obtaining ionized analytes; and
determining the content of boar taint compounds in the fat sample by subjecting the ionized analytes to mass spectrometry.

36. The method of claim 35, wherein the fat sample comes from an animal of the species sus scrofa.

37. The method of claim 35, wherein the fat sample is a backfat sample.

38. The method of claim 37, wherein the indole components comprise indole and/or skatole.

39. The method of claim 35, wherein extracting the boar taint compounds from the fat sample comprises liquid-liquid extraction using an extraction solvent.

40. (canceled)

41. The method of claim 39, wherein the liquid-liquid extraction comprises Salt Assisted Liquid-Liquid Extraction (SALLE), wherein the SALLE comprises:
homogenizing the fat sample in a brine solution;
adding the extraction solvent which is immiscible with the brine solution; and
transferring the boar taint compounds to the extraction solvent.

42. The method of claim 39, wherein the liquid-liquid extraction comprises Salt Assisted Liquid-Liquid Extraction (SALLE), wherein the SALLE comprises:
homogenizing the fat sample in a 2-phase system comprising a brine solution and the extraction solvent which is immiscible with the brine solution; and
transferring the boar taint compounds to the extraction solvent.

43.-50. (canceled)

51. The method of claim 35, further comprising adding an androstenone internal standard and an indole internal standard to the boar taint extract.

52. The method of claim 51, wherein the androstenone internal standard comprises androstenone-d4, androstenone-d5 or a C¹³-labeled androstenone.

53. The method of claim 51, wherein the indole internal standard comprises skatole-d3 or a C¹³-labeled skatole, and/or indole-d7 or a C¹³-labeled indole.

54. The method of claim 35, wherein the reaction solvent comprises a polar aprotic solvent.

55. The method of claim 35, wherein the strong base comprises at least one of sodium bis(trimethylsilyl)amide (NaHMDS), potassium bis(trimethylsilyl)amide (KHMDS) and lithium bis(trimethylsilyl)amide (LiHMDS).

56. The method of claim 35, wherein the organic solvent comprises at least one of THF, hexane, diethyl ether and methyl-ter-butyl ether.

57.-58. (canceled)

59. The method of claim 35, wherein the substrate is of general formula R—X, wherein:
R is aralkyl; or substituted aralkyl; and
X is Cl, Br or I.

60. The method of claim 35, wherein the base is an NaHMDS solution in THF and the substrate is 2,3,4,5,6-pentafluorobenzyl bromide or benzyl bromide.

61. The method of claim 35, wherein the polar aprotic solvent comprises at least one of acetone, DMF, DMSO and acetonitrile.

62.-64. (canceled)

65. The method of claim 35, wherein drying the solubilized analytes comprises removing the reaction solvent by evaporation at room temperature.

66.-67. (canceled)

68. The method of claim 35, wherein desorbing the dried analytes comprises indirectly heating the dried analytes with infra-red light having a wavelength between 800 and 1040 nm.

69. (canceled)

70. The method of claim 35, wherein ionizing the desorbed analytes comprises ionizing using a corona discharge.

71. The method of claim 35, wherein the mass spectrometry comprises tandem mass spectrometry.
